(19) United States
(12) Patent Application Publication
(10) Pub. No.: US 2023/0085786 A1
(43) Pub. Date: Mar. 23, 2023

(54) MULTI-STAGE MACHINE LEARNING TECHNIQUES FOR PROFILING HAIR AND USES THEREOF

(71) Applicant: The Joan and Irwin Jacobs Technion-Cornell Institute, New York, NY (US)

(72) Inventors: Tiffany ST. BERNARD, New York, NY (US); Kemar REID, Nashville, TN (US); Monte HANCOCK, Murray, KY (US)

(73) Assignee: THE JOAN AND IRWIN JACOBS TECHNION-CORNELL INSTITUTE, NEW YORK, NY (US)

(21) Appl. No.: 17/934,927

(22) Filed: Sep. 23, 2022

Related U.S. Application Data
(60) Provisional application No. 63/247,589, filed on Sep. 23, 2021.

(51) Int. Cl. G16H 50/20; G06N 5/02; G06N 20/20

(52) U.S. Cl. CPC G16H 50/20; G06N 5/02; G06N 20/20

(57) ABSTRACT

Techniques for generating recommendations using machine learning with respect to semantic concepts defined in a knowledge graph. A hair profile is determined for a user based on inputs related to the user. Determining the hair profile includes extracting attributes of the user from the inputs using natural language processing, computer vision, or both, and identifying respective nodes for the extracted attributes in the knowledge graph. The knowledge graph is created via machine learning using population data including hair-related data in order to identify relationships between semantic concepts represented by nodes of the knowledge graph. The nodes include discrete properties such as individual hair attributes, ingredients of products, or otherwise discrete characteristics of factors that may affect a user's hair or related health conditions. A generalized recommendation is generated based on the hair profile. A personalized recommendation may be generated based on the generalized recommendation and progress logged by the user.

### CROSS-REFERENCE TO RELATED APPLICATIONS

This application claims the benefit of U.S. Provisional Patent Application No. 63/247,589 filed on Sep. 23, 2021, the contents of which are hereby incorporated by reference.

### TECHNICAL FIELD

The present disclosure relates generally to machine learning, and more specifically to a multi-stage machine learning process for mapping hair attributes and profiling hair with respect to such mapped hair attributes in order to enrich hair and skin-related data.

### BACKGROUND

Many data biases are caused by a lack of contextualized, sufficiently granular data needed to effectively represent nuanced information. This problem is particularly acute for diverse groups that may have complex interdependent relationships. Attempting to use data lacking sufficient granularity therefore results in applying models to heavily biased datasets, thereby providing inaccurate results.

This lack of data granularity is a major issue in the hair health industry, which has historically lacked suitable healthcare guidance for various underrepresented groups and for which the depth of research into the effects of different hair care practices on such groups is shallow at best. As a result, the amount and granularity of data available for such groups is insufficient to provide appropriate guidance which meets the particular needs of individuals belonging to those groups.

Proper hair care plays an important role in both physical and mental health. Hair styling is an important component of grooming, which contributes significantly to self-esteem and confidence. Moreover, hair condition is linked both to skin conditions and broader health conditions such that providing ample hair-related data can also help evaluate other health-related conditions. Specifically, in addition to health-related benefits of hair such as protecting the scalp from sun damage, changes in appearance of hair can act as visual indicators of disease.

In addition to challenges in achieving stylistic and grooming goals which have harmful effects on mental health, the lack of availability of sufficient data has impeded spread of knowledge related to broader reaching health implications of hair products such as, but not limited to, cancers, fibroids, baldness, skin conditions, allergies, and other illnesses. Systemic biases in hair-related data have made diagnosing, researching, and treating such diseases more difficult.

Although some independent product developers and content creators have sought to provide solutions to address market needs that have not been addressed by mainstream hair product companies, the products and information offered by these independent entities tend to focus on hyper-focused niches that may not be suitable for all individuals and typically require that the user accurately understand the properties of their own hair. Additionally, the guidance provided by such content creators may create unrealistic goals and expectations for an individual's hair, which may actually have detrimental effects on the individual's hair and mental health despite any good intentions.

Some solutions for providing information include personalized hair quizzes attempting to provide personalized guidance on products and styles based on inputs ranging from hair type to goals. However, such quizzes lack a solid foundation in science due to the absence of sufficiently robust data and can therefore provide questionable or even dangerous recommendations. Additionally, some questions presented in these quizzes rely on the user knowing enough about their own hair to provide accurate answers, and therefore may be based on inaccurate inputs.

Consequently, it can be extremely difficult for consumers to determine which of these niche offerings are best for their specific needs. Using products and hair styling techniques which are not right for a particular individual, especially when the user does not fully understand how to properly utilize these products and techniques on their hair type, can and frequently does result in damage to hair. For example, follicular degeneration syndrome is a condition where a patient's hair follicles are damaged such that hair may interfere with hair growth. Although the exact causes of follicular degeneration syndrome are unknown, possible contributing factors that have been hypothesized include commonly used hair styling practices such as chemical straightening, relaxers, tight braids, heavy extensions, and using certain oils, gels, or pomades.

Some potentially harmful hair styling practices may be utilized in an effort to achieve Eurocentric beauty standards or to meet expectations of others that reject certain natural hairstyles in the short term but can cause long term damage to hair. For many individuals whose hair has been damaged by these hair styling practices, this damage has warped their understanding of hair condition such that those affected may not have a clear understanding of which hair conditions are healthy and which hair conditions are not. This, in turn, makes it difficult to tell whether certain products are styling techniques may be damaging one's hair.

The combined results of the historical issues noted above are a severe lack of hair data as well as systemic, self-reinforcing biases in data related to ethnic hair. More importantly, the relatively small amount of biased data that has been collected lacks granularity that would be needed to provide suitable and accurate hair care recommendations related to ethnic hair. Since healthy hair plays a significant role in mental as well as physical health, the lack of accurate granular data causes harm to the overall health of individuals and, in particular, individuals in minority communities.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for discretizing connections of semantically defined attributes using multi-stage machine learning. The method comprises: identifying a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices; mapping between semantic concepts of the plurality of semantic concepts, wherein mapping between the semantic concepts further comprises applying a first machine learning model trained to identify correlations between the plurality of semantic concepts with respect to the attributes and care practices identified within the population data; creating a knowledge graph including a plurality of nodes representing the plurality of semantic concepts and a plurality of edges connecting the plurality of nodes based on the mapping; applying a second machine learning model to visual content for a user in order to identify a subset of the attribute semantic concepts for the user, wherein the second machine learning model is trained to identify attribute semantic concepts of the plurality of attribute semantic concepts shown in the visual content; and querying the knowledge graph based on the identified subset of the attribute semantic concepts output for the second user, wherein the knowledge graph returns at least one care practice component semantic concept connected to the queried subset of the attribute semantic concepts.

Certain embodiments disclosed herein also include a non-transitory computer readable medium having stored thereon causing a processing circuitry to execute a process, the process comprising: identifying a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices; mapping between semantic concepts of the plurality of semantic concepts, wherein mapping between the semantic concepts further comprises applying a first machine learning model trained to identify correlations between the plurality of semantic concepts with respect to the attributes and care practices identified within the population data; creating a knowledge graph including a plurality of nodes representing the plurality of semantic concepts and a plurality of edges connecting the plurality of nodes based on the mapping; applying a second machine learning model to visual content for a user in order to identify a subset of the attribute semantic concepts for the user, wherein the second machine learning model is trained to identify attribute semantic concepts of the plurality of attribute semantic concepts shown in the visual content; and querying the knowledge graph based on the identified subset of the attribute semantic concepts output for the second user, wherein the knowledge graph returns at least one care practice component semantic concept connected to the queried subset of the attribute semantic concepts.

Certain embodiments disclosed herein also include a system for discretizing connections of semantically defined attributes using multi-stage machine learning. The system comprises: a processing circuitry; and a memory, the memory containing instructions that, when executed by the processing circuitry, configure the system to: identify a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices; map between semantic concepts of the plurality of semantic concepts, wherein the system is further configured to apply a first machine learning model trained to identify correlations between the plurality of semantic concepts with respect to the attributes and care practices identified within the population data; create a knowledge graph including a plurality of nodes representing the plurality of semantic concepts and a plurality of edges connecting the plurality of nodes based on the mapping; apply a second machine learning model to visual content for a user in order to identify a subset of the attribute semantic concepts for the user, wherein the second machine learning model is trained to identify attribute semantic concepts of the plurality of attribute semantic concepts shown in the visual content; and query the knowledge graph based on the identified subset of the attribute semantic concepts output for the second user, wherein the knowledge graph returns at least one care practice component semantic concept connected to the queried subset of the attribute semantic concepts.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe various disclosed embodiments.

FIG. 2 is a flowchart illustrating a method for generating recommendations using genome mapping according to an embodiment.

FIG. 3 is a flowchart illustrating a method for genome mapping with respect to discrete attributes and components of care practices according to an embodiment.

FIG. 4 is a flowchart illustrating a method for creating a personalized attributes profile according to an embodiment.

FIG. 5 is a schematic diagram of a genome cartographer according to an embodiment.

FIG. 6 is a flow diagram illustrating a process for extracting semantic concepts and relationships therebetween which may be utilized for creation of a knowledge graph according to another embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments include techniques for genome mapping and profiling with respect to attributes, particularly hair attributes using machine learning in a multi-stage process. In an embodiment, a knowledge graph including nodes and edges is created. The nodes of the knowledge graph include nodes representing semantic concepts which act as an ontology of attributes (e.g., hair attributes) as well as nodes representing discrete components of care practices (e.g., hair-related products, hair care practices, skin or other health condition products or practices, discrete portions thereof such as ingredients of products), combinations thereof, and the like. The edges represent connections between the nodes of the knowledge graph and may represent connections or relationships between, for example, certain hair attributes and ingredients of hair care products. The connections in the knowledge graph are determined at least partially using machine learning based on population data illustrating attributes of individuals within the population and care practices or portions thereof (e.g., products used for care practices) used by those individuals. At least some of the attributes are identified using a computer vision (CV) model trained to identify attributes associated with predefined semantic concepts based on visual content, for example images or videos showing individuals' hair.

In a further embodiment, a profile may be at least partially determined for a user based on visual content such as visual multimedia content showing the user. For example, a hair profile may be created based on images or videos showing the user's hair. Computer vision is performed on the visual content in order to extract hair attributes related to the semantic concepts of the knowledge graph. A hair profile including the extracted hair attributes is created.

Based on the profile and the knowledge graph, a generalized recommendation may be generated. The recommendation may be, but is not limited to, a recommendation to perform one or more care practices using certain discrete components such as to use one or more hair care products having certain ingredients. Specifically, products having ingredients which have positive connections to hair attributes for an individual having a particular hair profile can be identified using product identification rules with respect to the individual's hair profile and the knowledge graph. In some embodiments, the generalized recommendation may be utilized to generate a personalized recommendation based further on logged progress of the individual after using certain products, target hair care goals or styles, user preferences, or a combination thereof.

The disclosed embodiments provide techniques for classifying and contextualizing data by breaking data into more discrete components that impart additional information and detail to data via machine learning models, thereby decreasing bias and improving the accuracy of such data. Consequently, the accuracy of any results of using such data is increased. The disclosed embodiments further utilize multiple machine learning models in combination in order to produce data with higher granularity than would be produced by any one machine learning model individually.

More specifically, the disclosed embodiments utilize a multi-stage machine learning process including a stage used for creating a knowledge graph connecting semantic concepts representing profile attributes to discrete components of care practices (e.g., ingredients of hair care products used for hair care), and a stage used for determining a profile for a user based on user-provided content with respect to semantic concepts represented by nodes of the knowledge graph. The knowledge graph is created using machine learning and natural language processing to extract relationships between attributes and care practice components from unstructured text.

Once created, the knowledge graph can be queried with respect to profile attributes of a given user's profile in order to retrieve relevant information with respect to components represented as semantic concepts in the knowledge graph, for example information related to ingredients of products. Such relevant information can, in turn, be utilized for purposes such as, but not limited to, generating recommendations of products that would be suitable for the user based on the ingredients of those products, generating responses to questions submitted by the user (e.g., responses for an interactive chat program regarding the interactions between certain hair care products or ingredients and the user's hair), combinations thereof, and the like.

Additionally, when progress is logged in accordance with various disclosed embodiments, the logged progress may be used as feedback to improve the knowledge graph. Specifically, logged progress may be indicative that a certain connection in the knowledge graph is not accurate or is not always accurate. As a result, using the logged progress allows for updating the knowledge graph to more accurately reflect connections in the mapping.

In this regard, it has been identified that existing data relating hair to products is not sufficiently granular to effectively represent the effects of components of hair care products on discrete aspects of hair condition and, at best, offer general connections between broad categories of hair types and products which are “good” and “bad” for that hair type. Similar issues with granularity exist for skin conditions and other health-related conditions. By utilizing a first stage of machine learning to create the knowledge graph mapping discrete hair attributes to specific ingredients of hair care products and a second stage of machine learning to determine a hair profile using images of a user's hair, previously unknown and more granular connections between discrete ingredients and discrete hair attribute can be uncovered, which in turn can be utilized to provide more accurate insights related to hair and hair care products.

Further, the connections between discrete attributes and components of care practices can be leveraged to provide more accurate recommendations with respect to user's hair or other health goals. As an example, a user having certain hair attributes (e.g., wavy with wide waves) may wish to have their hair reach a certain length in a period of 12 months. Based on the user's existing hair profile with these attributes and the known connections between certain ingredients and hair length, personalized recommendations of products including suitable ingredients to reach the user's goals can be generated. These recommendations would, in turn, be more accurate than, for example, recommendations generated based only on associations between hair type and known products.

In addition to the technical advantages noted herein that allow for automating recommendations, the disclosed embodiments include techniques that can provide other benefits to users. More specifically, the disclosed embodiments provide more accurate recommendations for hair care and related health conditions of users which can utilize data provided by large numbers of individuals, thereby increasing the availability of information related to hair and skin care for people who historically have had difficulty finding appropriate information for their needs. Also, the improved granularity of the disclosed embodiments can be utilized to mitigate the effects of systemic bias that has become intertwined with the data.

Moreover, users do not require extensive familiarity with their own attributes in order to gain appropriate insights and recommendations using the disclosed techniques. Specifically, hair profiles may be created automatically using computer vision and without requiring further inputs from the user indicating information about their hair. As needed, a minimal amount of highly targeted follow up questions may be utilized to improve results. As a result, the disclosed embodiments can help with overcoming misunderstandings and lack of knowledge about hair condition which have become commonplace.

Additionally, the disclosed techniques can be integrated into social platforms in order to connect users with similar hair profiles as determined with respect to the discrete hair attributes, thereby allowing users to discuss their hair with other users who are likely to have the most similar experiences and may therefore be able to provide additional context for hair-related challenges. Because of the increased granularity of data used for mapping as described herein, potential connections between users can be determined more accurately, thereby improving user experiences. Thus, the disclosed data enrichment and mapping techniques can be used to aid in such efforts.

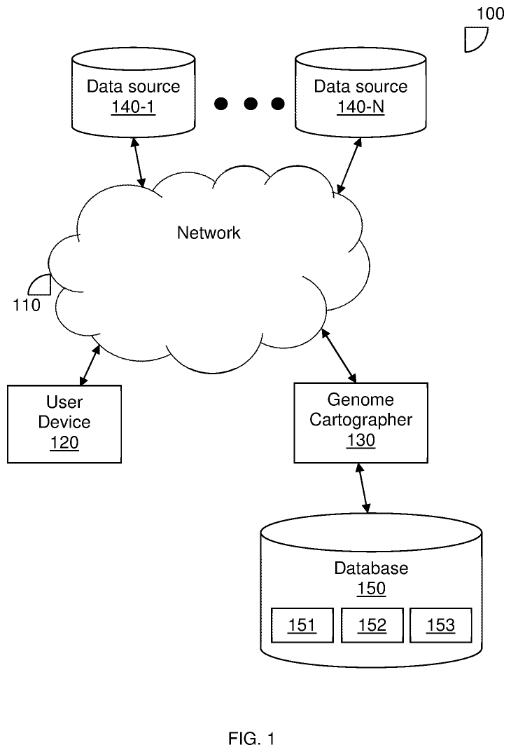

FIG. 1 shows an example network diagram 100 utilized to describe the various disclosed embodiments. In the example network diagram 100, a user device 120, a genome cartographer 130, and a plurality of databases 140-1 through 140-N (hereinafter referred to individually as a database 140 and collectively as databases 140, merely for simplicity purposes) communicate via a network 110. The network 110 may be, but is not limited to, a wireless, cellular, or wired network, a local area network (LAN), a wide area network (WAN), a metro area network (MAN), the Internet, the worldwide web (WWW), similar networks, and any combination thereof.

The user device (UD) 120 may be, but is not limited to, a personal computer, a laptop, a tablet computer, a smartphone, a wearable computing device, or any other device capable of receiving and displaying notifications. The user device 120 has one or more input/output interfaces (not shown) for receiving user inputs, displaying outputs (e.g., hair recommendations generated as described herein and/or other content related to hair recommendations such as videos or images used to demonstrate proper hair care practices), both, and the like.

The data sources 140 store data of many individuals (e.g., users or other subjects such as other humans or animals like cats or dogs) to be utilized for genome mapping and determining generalized inferences with respect to different kinds of hair. Such data may include, but is not limited to, images of various individuals' hair after using certain haircare products as well as textual data provided by those individuals, chemical models of users' hair, structural models of users' physical features (e.g., 3D models of users' hair), genetic models of users, behavioral data (e.g., social media posts collected from social media websites visited by users), combinations thereof, and the like. Such textual data may indicate information related to use of various haircare products by various individuals such as, but not limited to, products used, ingredients of those products, values representing amounts of each ingredient in a product (e.g., a relative amount such as a percent by volume or an absolute volume of a given ingredient), sensory observations by individuals after using certain haircare products, product reviews of haircare products, combinations thereof, and the like.

In some implementations, the user device 120 may receive user inputs indicating information such as, but not limited to, hair, skin, fur, or other health-related goals (e.g. stylistic goals, hair health goals, skin health goals, etc.), products used by a user of the user device 120 (or other subject), hair or other health-related condition progress (e.g., daily, weekly, or other periodic or irregular updates related to progress) for the user of the user device 120, experiences of the user of the user device 120 after using different care products or practices, reviews of care products by the user of the user device 120, combinations thereof, and the like. Such inputs may come in forms such as, but not limited to, text, selections (e.g., a selection made via radio button or check box), combinations thereof, and the like.

In various implementations, the user device 120 further includes one or more cameras (not shown) configured to capture images or other visual content (e.g., images or multimedia content including visual portions) reflecting hair condition, skin condition, other health conditions, and the like. As a non-limiting example, such visual content may include images or videos of a user's hair at various points in time used to track progress of hair condition.

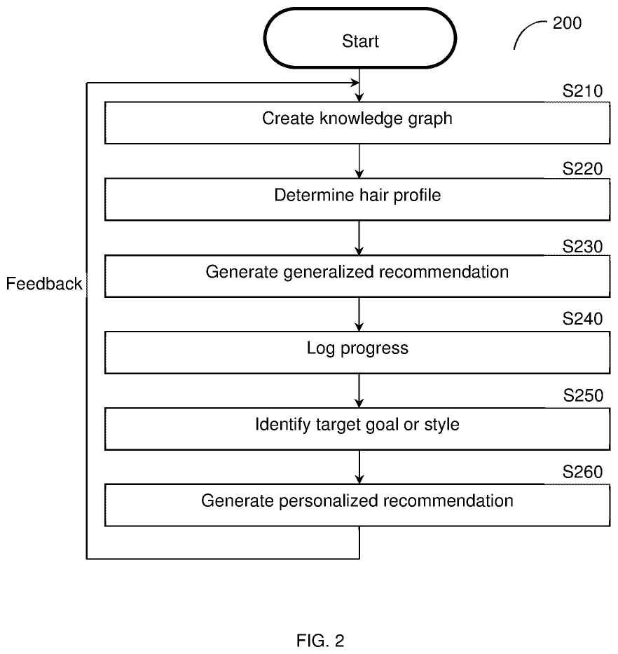

In an embodiment, the genome cartographer 130 is configured to map genomes and to generate recommendations as described herein, for example, as described below with respect to FIGS. 2-4. Specifically, a knowledge graph created as described herein includes nodes representing hair attributes, skin attributes, or other health-related attributes related to the human genome, thereby allowing for profiling an individual's genome with respect to these mapped attributes. In a further embodiment, the genome cartographer 130 is configured to map the genome with respect to hair attributes and ingredients of various haircare products in order to establish connections between hair conditions and ingredients.

To this end, in an embodiment, the genome cartographer 130 is configured to perform natural language processing on text indicative of interactions of ingredients of care products with users having different conditions (e.g., types of hair), image processing on images showing the user (e.g., the user's hair), or both, in order to extract features which can be used to map the user's hair with respect to the genome mapping. Mapping the user's hair with respect to a genome mapping as described herein, in turn, allows for deriving data describing the user's hair with a high degree of accuracy and granularity. Likewise, mapping skin or other health-related attributes using the techniques described herein allows for achieving similar accuracy and granularity for these other kinds of attributes.

The genome cartographer 130 is configured to store, in the database 150, a genome map 151, user tracking data 152, and previously generated recommendations 153. Data stored in the database 150 may be subsequently accessed in order to generate recommendations requested by users (e.g., by a user of the user device 120 or of other user devices, not shown).

In an embodiment, the genome cartographer 130 is configured to generate generalized recommendations for a user based on user inputs demonstrating features of the user's hair and the genome mapping. Alternatively, or collectively, the genome cartographer 130 is configured to generate personalized recommendations based further on hair targets (e.g., a target hair health goal, a target hair style goal, etc.). The recommendations may be generated in response to requests for recommendations from the user device 120 and may be based on data (e.g., images, user inputs, etc.) received from the user device 120.

It should be noted that the attribute data discussed with respect to FIG. 1 is described in the context of attributes of users (e.g., a user of the user device 120), but that the disclosed embodiments are equally applicable to other subjects. For example, a subject may be a non-user human or a non-human individual such as a pet (e.g., a dog or cat). To this end, when attributes of a non-human subject are analyzed, the discrete attributes may include characteristics of fur, claws, or other health-related traits of the non-human subject. At least some of the discrete attributes related to hair may be equally applicable as discrete attributes of an animal's fur.

FIG. 2 is a flowchart 200 illustrating a method for generating recommendations using genome mapping according to an embodiment.

At S210, genome mapping is performed in order to create a knowledge graph including connections between discrete attributes of users (e.g., attributes of hair profiles) and discrete components of care practices (e.g., discrete ingredients of hair care products used for hair treatment). More specifically, attribute semantic concepts representing possible discrete attributes (e.g., of hair profiles) are defined based on data (e.g., hair-related data) of various individuals, and the attribute semantic concepts are mapped to care practice component semantic concepts representing discrete components related to care practices for attributes such as ingredients which are components of hair care products or components of a treatment regimen.

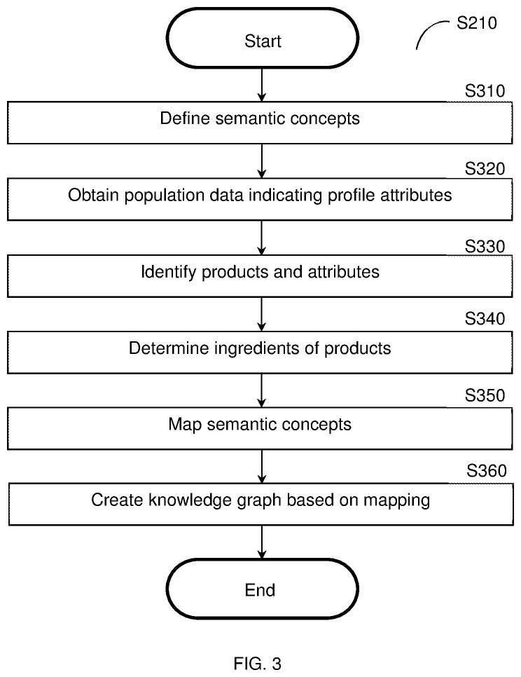

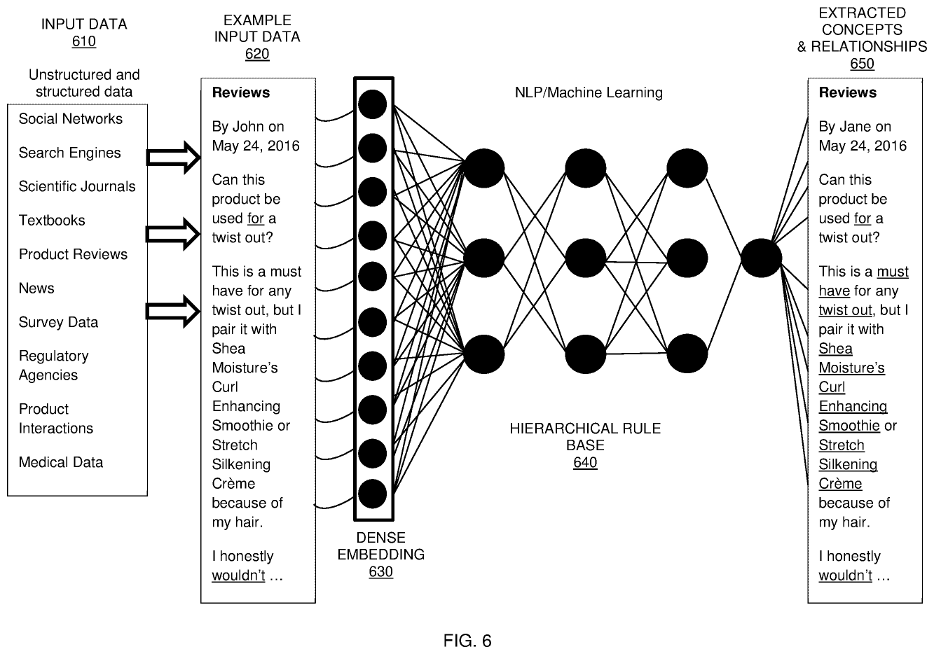

In an embodiment, genome mapping is performed as described with respect to FIG. 3. In another embodiment, the genome mapping is performed as described with respect to FIG. 6. FIG. 3 is a flowchart S210 illustrating a method for genome mapping with respect to discrete attributes and components of care practices according to an embodiment.

At S310, semantic concepts are defined. The semantic concepts represent discrete attributes to be used for profiling users' hair, skin, or other health-related conditions, and may be defined based on known attributes. Each semantic concept is a data object representing one or more terms which collectively represent a broader concept. Such terms may include terms directly representing concepts, synonyms for those terms, grammatical variations of words (e.g., different tenses, plural versus non plural, etc.), versions of terms in different languages, symbols or other markers which represent different concepts, and the like. In an embodiment, the semantic concepts provide an ontology defining various hair or other health-related attributes.

In an embodiment, the attributes represented by the attribute semantic concepts are properties associated with hair such as, but not limited to, hair types defined in a predetermined classification scheme (e.g., the Andre Walker Hair Typing System, also known as “The Hair Chart”) or portions thereof (e.g., coiled), porosity, density, texture, elasticity, thinning status, other characteristics of hair, and degree of severity or other descriptors of conditions (e.g., loosely).

As a non-limiting example, the attribute semantic concepts may be defined with respect to properties of different hair types such as 1a (i.e., straight [fine] per The Hair Chart), 1b (i.e., straight [medium]), 1c (i.e., straight [coarse]), 2a (i.e., wavy [loose waves]), and the like. The attribute semantic concepts may further represent hair characteristics such as soft, shiny, oily, volume, full, body, curl, waves, thick, thin, coiled, tight, loose, various patterns (e.g., “S” pattern, “o”-shaped pattern, etc.), different lengths (e.g., ranges or categories of lengths), variations thereof (e.g., synonyms, different tense versions of the same word, etc.), and the like.

By defining types of hair or other conditions not just based on broad classification but based on specific, discrete attributes (i.e., the properties represented by semantic concepts), a user's condition may be profiled with higher granularity, thereby improving the accuracy of results (e.g., recommendations) of using profiles created with respect to these discrete attributes as described herein. Further, mapping connections between discrete hair attributes and discrete components of care practices such as specific ingredients of hair care products as described herein allows for more accurately identifying connections between certain hair care products and different kinds of hair, thereby providing a higher granularity knowledge graph which can be utilized to more accurately generate recommendations based on a user's hair.

In a further embodiment, the semantic concepts also include semantic concepts defined with respect to discrete components of care practices or portions thereof such as products, styles, treatments, techniques, regimens, and the like. In this regard, it is noted that certain hair care practices can, either alone or in combination with certain ingredients, damage or otherwise permanently alter hair attributes. Accordingly, including hair care practices among the semantic concepts allows for mapping such connections, thereby allowing for uncovering relationships between different hair care practices and their effects on hair attributes. Additionally, like for hair care products, discretizing hair care practices into discrete components to be represented by semantic concepts provides more granular information related to connections with hair attributes, thereby improving accuracy when using the knowledge graph.

Moreover, semantic concepts for such hair care practices may be defined with respect to discrete components of the hair care practices, thereby allowing for mapping these discrete components of hair care practices to discrete hair attributes like the mapping of discrete product components to discrete hair attributes. As a non-limiting example, semantic concepts related to a particular hair care treatment may represent concepts such as whether the treatment involves heat, tools used for the treatment (e.g., a blow dryer, hot comb, or curling iron), scientific characteristics of tools (e.g., whether the tool uses heat, whether the tool mechanically works the hair, etc.), whether the treatment involves chemical treatment, whether hair coloring is used, combinations thereof, and the like. In some embodiments, such mapping for discrete components of hair care practices may be used in addition to or instead of the mapping for ingredients of hair care products.

In some embodiments, the semantic concepts may further include semantic concepts defined with respect to skin conditions or other health conditions (e.g., health conditions that are not strictly or directly related to appearance or structure of the hair itself). Semantic concepts related to skin condition may define discrete components of scalp conditions such as, but not limited to, irritated, flaky, bumps, rashes, and the like. Defining semantic concepts for skin or other health conditions allows for uncovering indirect connections between ingredients and such health conditions through hair attributes and breaking those conditions into discrete components also allows for improving granularity of connections as discussed above.

In yet a further embodiment, the semantic concepts may also include semantic concepts defined with respect to external factors such as nutrition or general wellness of individuals (e.g., hair supplements prescribed to the patient, stress levels, etc.), environmental factors, pets (e.g., pets owned by the user or that the user is otherwise in regular contact with), combinations thereof, and the like. The environmental factors may include, but are not limited to, sun exposure, humidity levels, smog, or pollution levels in different locations where individuals may reside, exposure to smoke, hardness of water in different locations where individuals may reside, combinations thereof, and the like. Mapping semantic concepts representing external factors allows for further uncovering interactions of such external factors with hair attributes and, moreover, how different care practices intersect with such external factors in order to affect hair attributes.

In yet another embodiment, the semantic concepts related to ingredients of care products may further include semantic concepts defined with respect to ingredient functions, i.e., the roles certain ingredients play as part of a broader product. As non-limiting examples, such ingredient function semantic concepts may include fragrance, detangling, conditioning, and the like. In this regard, it is noted that certain ingredients in products are included for particular purposes, and that these purposes may relate to specific types of attributes. Defining semantic concepts with respect to these ingredient functions therefore allows for deriving additional contextual information related to the connections between certain ingredients and their effects on hair or other attributes. Moreover, such ingredients may have specific chemical functions that can be unearthed by defining semantic concepts with respect to these functions.

At S320, data related to a population including various individuals having different hair or other health-related attributes is obtained. The population data indicates attributes of the individuals of the population as well as care practices used by those individuals (e.g., use of certain hair care products). Further, the population data indicates (explicitly and/or implicitly) potential connections between such attributes and hair care products.

The population data may include, but is not limited to, inputs indicating known associations between ingredients and hair attributes, user-generated content scraped from data sources available via the Internet (e.g., the data sources 140, FIG. 1), scientific journals, dermatological databases, combinations thereof, and the like. The user-generated content may include, for example, user reviews, comments, behavioral data such as social media posts, and the like. The inputs reflect effects of using different care practices (e.g., treating hair using different products) on individuals having different attributes (e.g., different hair attributes).

Potential connections between a given hair attribute and a given ingredient in the population data may be identified based on, for example, the distance between words representing the attribute and the ingredient within text, connecting words indicating causality (e.g., “cause,” “effect,” “made,” “did,” “affect,” “relationship,” “connection,” “link,” variations thereof, etc.), both, and the like. Moreover, whether a given connection is positive (i.e., indicating that the product had a positive effect on an individual's hair) or negative (e.g., damaged, or otherwise hurt the appearance of the individual's hair) may be determined based on the presence of certain predetermined positive or negative terms (e.g., “feels good,” “feels bad,” “damaged,” “improved,” “hurt,” “”lost,” etc.) in association with certain hair attributes in population data. Whether there is a connection and whether a connection is positive or negative can be learned via machine learning.

At S330, instances of attributes (e.g., hair attributes) and of care practices or portions thereof (e.g., products used to care for hair) are identified in the population data. The attributes may be identified by matching between portions of the population data and the terms represented in the semantic concepts. The hair care products may be identified, for example, based on a predetermined list of hair care products.

At optional S340, discrete components of the care practices or portions thereof (e.g., ingredients of hair care products) identified at S330 are determined. In an embodiment, S340 includes retrieving data reflecting ingredients identified at S330 and identifying discrete components of such lists representing ingredients. Such data reflecting ingredients may include, but are not limited to, product descriptions, ingredient lists, directions, labels, product specification pages, materials safety data sheets, and the like. Alternatively, the ingredients of each product may be included in predefined ingredient lists associated with respective products of the product list.

In a further embodiment, S340 includes retrieving data related to relative or absolute amounts of ingredients (e.g., a percent by volume or other amount of ingredient in a given volume of a product). Such ingredient amount data may be defined as semantic concepts, thereby allowing for connecting different amounts of ingredients to different attributes in order to provide additional granularity about the effects of certain ingredients on hair attributes or other health-related conditions.

Additionally, such information may be utilized to reverse engineer a product which will likely have beneficial effects on users having certain attributes or conditions by allowing for identifying how different relative amounts of ingredients affect hair or other health conditions using connections in the mapping between semantic concepts. More specifically, the knowledge graph may be accessed with respect to one or more desired hair or skin attributes to identify ingredients and, in particular, relative amounts of those ingredients, which will likely result in the desired hair or skin attributes or otherwise have a positive effect on a user having those attributes. To this end, the mapping between semantic concepts representing the desired attributes and semantic concepts representing the ingredients and the amounts of those ingredients may be analyzed to identify which ingredients and what amounts of each ingredient should be included in a product for the desired attributes. This information can be combined to provide a suggested product defined with respect to amounts of different ingredients, for example, a percent by volume of each ingredient to be included.

At S350, the semantic concepts are mapped to each other. In an embodiment, S350 includes using natural language processing techniques and, specifically, applying a machine learning model trained to identify correlations between semantic concepts indicated in text. Such natural language processing may unearth connections, for example, between ingredients in hair products or other discrete aspects of hair care practices with hair or other health attributes. In this regard, it is noted that connections between hair attributes and ingredients are not typically indicated explicitly in text, and that relationships between hair attributes and certain products do not necessarily imply causal relationships between hair attributes and specific ingredients of those products. Thus, using machine learning allows for identifying connections between these attributes and ingredients via implicit relationships even when connections are not explicitly expressed. Similarly, skin or other health conditions may exhibit causal relationships with particular subcomponents of certain skincare or healthcare practices which might not be explicitly indicated in text, although relationships between the conditions and the practices themselves may be indicated explicitly. Like for hair attributes and particular ingredients, causal relationships between discrete attributes in general may be discovered with discrete components of other care practices using machine learning as discussed herein.

In a further embodiment, S350 includes determining strengths of the connections to be used for the mapping. Such strengths of connections may be expressed via, for example, weights, and may be determined based on a number of connections identified between a given ingredient and a given hair attribute via the population data, degrees of causality indicated by connecting words, combinations thereof, and the like.

In an embodiment, S350 may include determining whether each connection between a component of a care practice or portion thereof (e.g., an ingredient) and a user attribute (e.g., a hair attribute) is a positive connection or a negative connection. A positive connection may occur when the population data indicates that an ingredient has a positive effect on a hair attribute (i.e., improves the look or feel) indicated by, for example, predetermined positive language in association with the ingredient. Likewise, a negative connection may occur when the population data indicates that an ingredient has a negative effect on the hair attribute (e.g., causes damage, causes predetermined undesirable changes in appearance, etc.). In some implementations, a value indicating a positive or negative connection may be associated with the edge. Such positive or negative connections in the mapping may be utilized when, for example generating recommendations (e.g., by recommending hair care products and practices that will likely have a positive effect on a user's hair and avoiding hair care products and practices that will likely have a negative effect on a user's hair.

In some embodiments, only connections having strengths above a threshold (e.g., having at least a threshold weight or number of connections identified in the population data) are included in the mapping. Determining strengths of connections allows for filtering out connections that are tenuous, for example, connections which may indicate some degree of correlation but not causality. In this regard, it has been identified that certain “common knowledge” connections between hair attributes and certain products or ingredients may be based on misunderstandings and other systemic gaps in knowledge noted above which may be reflected in user-generated content. Filtering out connections that are not sufficiently strong as described herein allows for avoiding perpetuating or otherwise reinforcing these misunderstandings.

In this regard, it has been identified that, by breaking down definitions of hair profiles into discrete attributes and breaking down hair products into ingredients, machine learning models can be trained to identify connections between instances of such discrete attributes and ingredients represented in textual content. More specifically, relationships between hair attributes and products containing certain ingredients can be learned via machine learning, and cumulative effects of using products containing different ingredients can be leveraged to obtain a model indicating connections between the hair attributes and specific ingredients. This, in turn, provides higher granularity data with respect to connections between hair care practices and different kinds of hair.

At S360, a knowledge graph is creating based on the mapping. The knowledge graph includes nodes and edges connecting the nodes. The nodes at least include nodes representing the attribute semantic concepts and nodes representing the care practice component semantic concepts (e.g., semantic concepts representing ingredients). The edges represent connections between the attributes and discrete components of care practices (e.g., between hair attributes and particular ingredients of products used to treat hair) uncovered during the mapping using machine learning as noted above. In some embodiments, each edge may further be associated with metadata indicating information about the edges such as weights, whether an edge represents a positive connection or a negative connection, and the like.

In some embodiments, the nodes may further include nodes representing skin or other health conditions. As noted above, hair condition relates to several other health-related conditions and, in particular, skin condition. Moreover, due to this relationship, ingredients used in hair products can be connected to certain skin or other health conditions. Thus, deeper effects of hair product ingredients on other health conditions may be uncovered based on indirect connections from ingredients to hair attributes and from hair attributes to other health conditions. Adding these connections to the knowledge graph therefore allows for leveraging these indirect connections in order to further research into medical conditions related to hair and hair care products, provide personalized recommendations based further on more general health goals, and the like. As a non-limiting example, it may be identified that paraphenylenediamine, which is a common allergen used in many permanent hair dyes, is connected to dermatitis.

Additionally, skin or other health conditions may similarly demonstrate connections to particular components of care practices when those conditions are broken down into discrete attributes. Thus, applying the disclosed embodiments to attributes of other health conditions allows for improving granularity of connections between those conditions and the care practices applied for them.

Returning to FIG. 2, at S220, a profile is determined for a user. The profile is defined with respect to semantic concepts represented in the knowledge graph and, specifically, with respect to attributes represented by respective semantic concepts of nodes in the knowledge graph. In an embodiment, determining the profile at least includes applying a machine learning model trained to identify discrete attributes among the semantic concepts in visual content (e.g., images, videos, etc.) such as, but not limited to, multimedia content including visual portions.

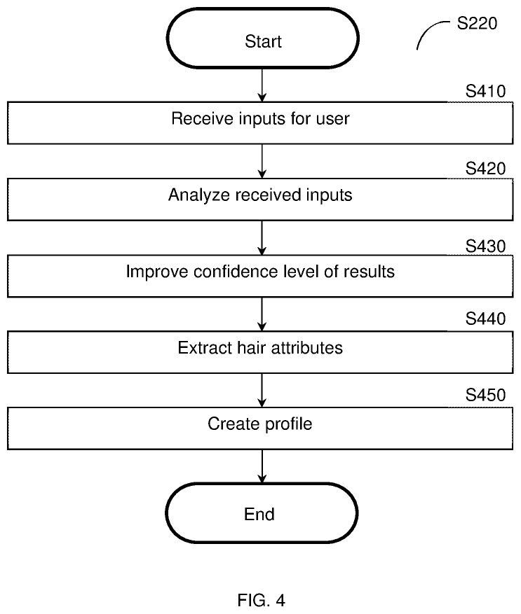

In an embodiment, the profile is created as described with respect to FIG. 4. FIG. 4 is a flowchart S220 illustrating a method for creating a personalized attributes profile according to an embodiment.

At S410, inputs related to the semantic concepts represented by nodes in the knowledge graph are received for a given user. The received inputs provide information about a particular user's hair, health or other conditions, environmental factors, combinations thereof, and other information that relates to semantic concepts which might be represented within the knowledge graph. To this end, such inputs may include, but are not limited to, visual multimedia content depicting the user's hair, quiz answers (e.g., inputs provided via a user interface in response to one or more questions directed to the user), health information (e.g., health conditions of the user, diet, or nutrition, etc.), environmental or other external factors (e.g., geographic location), chemical models, three-dimensional (3D) models, genetic models, combinations thereof, and the like. The visual multimedia content may be, but is not limited to, images, videos, combinations thereof, portions thereof, and the like.

In some embodiments, the inputs may include models of the users' health-related characteristics. As non-limiting examples, such inputs may include chemical models of users' hair, structural models of users' physical features (e.g., 3D models of users' hair), genetic models of users, and the like. These models may contain values representing the user's physical and biological properties which may correspond to semantic concepts represented in the knowledge graph.

Alternatively or additionally, the inputs may include behavioral data such as, but not limited to, contents of social media posts (e.g., text, pictures, emoji or other icons, etc.) which may be indicative of semantic concepts which may be applicable to each user, a preference a user has with respect to one or more semantic concepts (e.g., likes, dislikes, etc.), or both.

At S420, the inputs received for the user are analyzed. When the inputs include textual data (e.g., data representing quiz results, health information, or external factors), such textual data may be analyzed using natural language processing in order to identify potential concepts indicated therein. When the inputs include models (e.g., chemical, structural, or genetic models), such models are analyzed using respective algorithms designed for analyzing such models. When the inputs include visual content (e.g., visual multimedia content including at least some portion of visual content), the visual content is analyzed using computer vision (CV). To this end, in an embodiment, S420 includes applying a machine learning model trained to identify predefined attributes (e.g., hair attributes represented by the semantic concepts in the knowledge graph) in visual multimedia content.

In an embodiment, the machine learning model may be trained using supervised machine learning by applying a machine learning algorithm to labeled visual content having labels indicating different hair attributes shown in the visual content. In a further embodiment, the labeled training set may be created based on user inputs for different training visual content. The user inputs may be determined, for example, based on tags provided by users.

As noted above, the inputs may include models of physical and/or biological characteristics of users. To this end, in some embodiments, S420 may include analyzing such models in order to extract data which may be related to the semantic concepts represented in the knowledge graph which can be utilized in order to determine which attributes of the user are present in those models as defined with respect to the semantic concepts represented in the knowledge graph. To this end, different semantic concepts may be associated with different potential results of such model analysis (e.g., potential values representing aspects of chemical, structural, or genetic models), and the semantic concepts reflected in a given model may be determined by analyzing the model with respect to those associations. The associations may be predefined explicitly, or may be defined using machine learning.

Also noted above, the inputs may include behavioral data such as social media post contents. To this end, in some embodiments, S420 may further include analyzing such behavioral data in order to determine additional information with respect to the semantic concepts which may be applicable to the user for which a hair profile is being created. The analysis may further include applying a preference engine to determine and register user preferences based on such behavioral data.

At optional S430, additional user inputs may be requested and analyzed to improve the confidence level of the results of the computer vision. In an embodiment, S430 may be performed when the confidence level for one or more attributes is below a threshold. The request may indicate follow up questions for the user, where responses to the questions can be analyzed to confirm or reject hair attributes identified via the computer vision. This follow up can be used to improve the accuracy of the computer vision, thereby providing a more accurate hair profile for the user.

At S440, attributes of a user (e.g., hair attributes) are extracted based on the analysis of the inputs received for the user. The extracted hair attributes are defined by semantic attributes represented in a knowledge graph (e.g., the knowledge graph created as described with respect to FIG. 3).

In an embodiment, the semantic concepts used for creating a personalized attributes profile and generating recommendations therefore further include semantic concepts related to effects of different hair treatments (i.e., treatments using certain hair products, hair styling techniques, or a combination thereof). Such effect-defining semantic concepts may include, but are not limited to, growth rates, lengths, indicators of esteem (e.g., as defined with respect to the Bankead/Johnson Hair Esteem Scale), indicators of pain or discomfort, combinations thereof, and the like. At least some of the semantic concepts may be defined during genome mapping as described above with respect to S310.

At S450, a profile is created for the user based on the extracted attributes. When the profile is a hair profile, the hair profile includes the attributes which at least indicate characteristics of the user's hair (e.g., porosity, density, texture, hair type, etc.). More specifically, the hair profile includes attributes represented in a knowledge graph including at least connections between hair attributes and ingredients as described above.

Returning to FIG. 2, at S230, a generalized recommendation is generated for the user based on the user's profile. The generalized recommendation is a recommendation for a user having the kind of condition or conditions reflected in the user's profile. The recommendation may suggest one or more products, care practices, or both, for improving the attributes of the user and/or maintaining healthy condition of the user.

In an embodiment, the generalized recommendation may be generated by identifying appropriate care practices with respect to the user's profile and connections between attributes of the user's profile with other nodes of the knowledge graph. The care practices may include, but are not limited to, hair care practices such as using certain hair care products, using or not using certain hair care tools, performing certain styling techniques, combinations thereof, and the like. As a non-limiting example, an appropriate hair care practice may be to use a particular hair care product having ingredients with positive connections to hair attributes of the user's hair profile (i.e., ingredients known to have positive effects on the user's hair attributes). The recommendation may further indicate settings for tools to be used such as, but not limited to, temperature settings or comb physics.

When the recommendation would include recommendation of a hair care product and multiple potential appropriate products are identified, the recommendation may indicate alternative treatment plans using different products or may recommend the product having the strongest relationship with the user's hair profile (e.g., the product having the most ingredients connected to attributes of the user, the product having the ingredients with the strongest connections to attributes of the user, etc.). Likewise, the recommendation may be selected from among non-health related aspects of the products such as user preferences, prices of certain products or tools needed for the care practices, availability of certain products or tools in a given geographic location (e.g., availability within a country or region a user lives in), combinations thereof, and the like.

In some implementations, the recommendation may further include recommended content for the user. As a non-limiting example, such recommended content may be a tutorial video or article demonstrating how to use a particular hair care product or how to perform a given hair styling technique. The recommended content may be predetermined content associated with different semantic concepts represented in the knowledge graph, may be content recommended by users having the same hair attributes or goals, both, and the like.

In another embodiment, the recommendations may indicate one or more health disorders of the user. As noted above, the semantic concepts represented in the knowledge graph may include more general (i.e., not directly hair-related) health attributes such as skin conditions. Accordingly, using the knowledge graph created as described herein, potential health disorders demonstrated through specific combinations of health attributes can be determined using diagnostic rules defining predetermined potential combinations of health attributes indicative of particular health disorders. Alternatively, or collectively, the recommendations may include recommendations to have such health disorders formally diagnosed by a doctor and/or treated.

At S240, progress of the user is logged after using one or more care products or practices (e.g., styling techniques). In an embodiment, S240 includes receiving user inputs, images, or both, from a user device of the user (e.g., the user device 120, FIG. 1). Such user inputs and/or images demonstrate effects of using the products or care practices on the individual user. The user inputs may include textual inputs such as, but not limited to, diary entries, product reviews, comments, combinations thereof, and the like.

In an embodiment, S240 may further include providing questions to the user (e.g., via a user interface) and receiving user inputs in response to the questions. The questions may be targeted based on the user's profile and, specifically, may use terms corresponding to discrete components represented by the semantic concepts in order to obtain specific data related to potential connections between various care practices or health conditions and attributes, which in turn can be used as feedback to improve the accuracy of the mapping in the knowledge graph.

At optional S250, a target is identified for the user. The target may be, but is not limited to, a target health goal (e.g., growing hair to a certain length, achieving a certain color or porosity, etc.), a target style goal (e.g., hairstyles such as box braids, Bantu knots, silk press, variations thereof, etc.), and the like. In an embodiment, S250 includes receiving data indicating content viewed by the user during an exploration and data indicating user interactions with at least a portion of the viewed content. In a further embodiment, the target goal or style is determined based on portions of the content with which the user interacted.

In yet a further embodiment, one or more interaction rules may define which types of interactions are used to determine the target goal or style when the target is not provided explicitly via user inputs indicating a particular target and may further define requirements for using such interactions (e.g., only using interactions of at least a predetermined time length such as viewing at least 1 minute of a video). As a non-limiting example, if the user clicked on a video showing a person with Bantu knots and viewed the video for at least one minute, it may be determined that Bantu knots are a potential target hair style for the user and Bantu knots may be identified as the target.

At S260, a personalized recommendation is generated for the user based on the generalized recommendation and the logged process. In an embodiment, the personalized recommendation is for improving health, appearance, or both, of the user's hair, and is determined based on reactions of the user's hair to different hair care practices indicated in the logged progress. To this end, S260 may include performing natural language processing on textual content indicating progress of the user and hair care practices used by the user, performing image processing on images showing the user's hair, or both, in order to determine how the products and/or techniques used by the user have affected the condition of the user's hair as compared to the known connections reflected in the genome mapping.

In an embodiment, the personalized recommendation is further based on the target identified at S250. Specifically, the personalized recommendation may suggest care practices associated with attributes of the target. As a non-limiting example, when the target is a hair style that requires hair at least of a certain length, the personalized recommendation may suggest using products having ingredients associated with longer hair lengths for the user's hair profile.

As noted above, the knowledge graph may include semantic concepts representing different amounts of ingredients mapped to semantic concepts representing certain hair or other health-related attributes, or may otherwise include amount information in connection with semantic concepts representing different ingredients. In some embodiments, S260 may further include generating a recommendation for a product based on such amounts of ingredients. As a non-limiting example, a recommendation for a product having 10% coconut oil, 20% biotin, 30% Lauryl Glucoside, and 40% carrying or other ingredients (e.g., including non-active ingredients such as solvents).

In another embodiment, the personalized recommendation may be based further on one or more preferences of the user. To this end, in some embodiments, S260 may include applying a preference engine to potential recommendations in order to determine which recommendations are in line with the user's preferences. The preference engine accesses information about the user's preferences and is configured to determine whether a given recommendation matches the user's preferences. As a non-limiting example, the preference engine may access information indicating that the user dislikes coconut oil such that any recommendations including use of products including coconut oil are determined not to be in line with the user's preferences and excluded from the personalized recommendations. The preference engine may be trained using machine learning (e.g., based on information collected about the user such as social media posts indicating ingredients the user likes and dislikes) or preconfigured with explicitly provided user preferences.

In an embodiment, the generalized recommendation, the personalized recommendation, or both, may be utilized to treat one or more conditions of the user as defined with respect to attributes represented by the attribute semantic concepts. To this end, S230, S260, or both, may include treating the conditions of the user based on care practices including the discrete components as included in the respective recommendations. The treatment may include, but is not limited to, prescribing a care practice regimen including the recommended care practices, causing execution of the application of one or more care products including recommended ingredients, providing content demonstrating proper care practice routines based on the recommended care practices, combinations thereof, and the like.

In another embodiment, the process described with respect to FIG. 2 may further include generating one or more predictions using the knowledge graph. In particular, S230, S260, or both, may further include generating such predictions and determining the respective recommendations based on the predictions. Alternatively, a prediction may be generated without generating a recommendation in some embodiments. The prediction may be, but is not necessarily limited to, a prediction of one or more future discrete attributes for the user. Such predicted future discrete attributes may be determined based on the connections in the knowledge graph related to one or more discrete components of care practices to be followed by the subject, a current set of discrete attributes for the subject, both, and the like.

In the embodiment shown in FIG. 2, execution may continue with S210 after user progress is logged. This allows for using the logged progress as feedback to improve the knowledge graph. Further although FIG. 2 depicts performing the entire method at each iteration, only a part of the method may be performed in subsequent iterations (for example, the knowledge graph may be updated without performing other steps) without departing from the scope of the disclosure. In other embodiments (not shown), execution may terminate after S260.

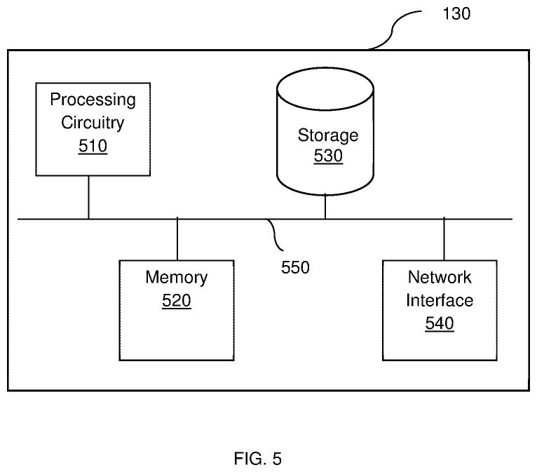

FIG. 5 is an example schematic diagram of a genome cartographer 130 according to an embodiment. The genome cartographer 130 includes a processing circuitry 510 coupled to a memory 520, a storage 530, and a network interface 540. In an embodiment, the components of the genome cartographer 130 may be communicatively connected via a bus 550.

The processing circuitry 510 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 520 may be volatile (e.g., random access memory, etc.), non-volatile (e.g., read only memory, flash memory, etc.), or a combination thereof.

In one configuration, software for implementing one or more embodiments disclosed herein may be stored in the storage 530. In another configuration, the memory 520 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 510, cause the processing circuitry 510 to perform the various processes described herein.

The storage 530 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 540 allows the genome cartographer 130 to communicate with, for example, the user device 120 the data sources 140, the database 150, or a combination thereof.

It should be understood that the embodiments described herein are not limited to the specific architecture illustrated in FIG. 5, and other architectures may be equally used without departing from the scope of the disclosed embodiments. Further, although the various disclosed methods are described as all being performed by the system 130, it should be noted that different systems may perform different methods without departing from the scope of the disclosure. As a non-limiting example, in some implementations, the genome mapping described with respect to FIG. 3 may be performed by a first system configured as depicted in FIG. 5 and the methods of FIGS. 2 and 4 may be performed by a second system that is similarly configured as depicted in FIG. 5.

Another process for creating a knowledge graph which may be utilized in accordance with various disclosed embodiments is now described. Portions of this process are depicted and will be described with respect to FIG. 6. In accordance with various disclosed embodiments, a knowledge engine may utilize such a process in order to draw connections between products, ingredients, brand person preferences, product reviews, and other aspects of attributes, care practices, or related contextual information.

In an embodiment, information about a user's care situation (e.g., haircare situation) as well as a target (e.g., a goal, for example as described with respect to discrete attributes as discussed above) of the user are ingested, and a personalized recommendation is generated based on predetermined expert knowledge such that the personalized recommendation is tailored to achieve the target. Generating the recommendation may further include performing an assessment of a current condition of the user (e.g., as defined with respect to discrete attributes as discussed above), analyzing an existing care practice of the user (e.g., a haircare regimen using discrete haircare-related components of haircare practices), a suggested process for achieving the target (e.g., known expert knowledge for achieving a particular combination of attributes), and a predetermined description of predicted results of following the recommended care practice.

In this regard, it is noted that it is desirable for outputs of an automated system creating and using knowledge graphs as described herein to be user-accommodating, that is, to produce results which are succinctly expressed in natural language, using terms which are understandable to non-experts, and which can replicate the look and feel of mature work product produced by a human being. Utilizing artificial intelligence (AI) in order to model targets with respect to a knowledge graph in order to generate recommendations therefore provides a process which is able to address both the mathematical aspects of data preparation and the semantic processing needed to communicate results in human language.

To this end, in an embodiment, AI technology is introduced into the architecture of a recommender system at multiple points. In such an implementation, trainable image processing and semantic feature extraction allows for supporting extraction, selection, and enhancement of features. At the same time, knowledge-based inferencing allows for supporting diagnosis process planning, and recommendation generation.

In an embodiment, a system includes multiple software routines that run under the direction of a main routine. In a further embodiment, the processing is divided into multiple stages such as, but not limited to, feature assembly, triage, a first stage of inferencing, product analysis, a second stage of inferencing, and adjudication. In yet a further embodiment, these stages are performed sequentially starting with feature assembly, then triage, then the first stage of inferencing, then product analysis, then the second stage of inferencing, and finally adjudication. It should be noted that other stages may be implemented without departing from the scope of this embodiment.

The feature assembly may include the application of multi-dimensional scaling to reduce the data complexity for subsequent inferencing.

The inferencing for triage and recommendation building may be performed using a hierarchical knowledge-based expert system. This is a set of trainable heuristics which select and evaluate evidence in order to determine what information (“snippets”) should be included in the recommendation, based upon their specific presentation as indicated by the collected feature data.

After feature extraction, rules are applied to the resulting feature vectors in order to compute biases for and against the inclusion of information snippets to be included in the recommendation. In an embodiment, biases are aggregated from beliefs and disbeliefs using an aggregation rule. In a further embodiment, the rules adjudicate evidence using a bias-based aggregation rule, for example as summarized in Equation 1 below:

$${B}_{l} ( {β}_{1 , l} , … , {β}_{N , 1} , {δ}_{1 , l} , … , {δ}_{M , 1} ) = 1 - ( \frac{{\prod }_{k = 1}^{N} ( 1 - {β}_{kl} )}{{\prod }_{j = 1}^{M} ( 1 - {δ}_{jl} )} ) \qquad Equation 1$$

In Equation 1, βₖₗ≥0 is evidence inferred from features that information snippet 1 should be included in the recommendation, while δⱼₗ≥0 is evidence that snippet 1 should not be included. The final determination is based upon the aggregated evidence, quantified by βₗ.

In an embodiment, the process for generating a recommendation uses a hierarchical inferencing with the following stages:

In stage 1 (feature assembly), an intake survey is performed with inputs from a user, visual content (e.g., an image) is uploaded, and image processing is performed in order to extract image features from the uploaded visual content.

In stage 2 (triage), a case instance is classified. In an embodiment, the classification may be performed using a bias-based expert system approach which models a problem joint-probability distribution in a computationally-efficient manner that avoids the shortcomings of other inferencing schemes (e.g., Dempster-Shafer Theory, Bayesian Belief Nets, etc.). The bias-based expert system approach is also robust to missing features, which is a common occurrence that is a challenge for regression methods such as neural networks.

In stages 3-5, the case instance classified during triage is passed to an appropriate rules cluster which is part of the processing expert system. Bias-based reasoning is used to select a care practice (e.g., a regimen) for the recommendation. To this end, appropriate care practices (e.g., use of particular products) are selected which will allow for meeting the target or other concerns of the user. In various embodiments, natural language processing (NLP) is utilized to create target-to-product mapping tables which map known concerns or targets to care practices or portions thereof (e.g., use of certain products) which address those concerns or help meet those targets. The recommendations may further be optimized using preferences of the user, health concerns (e.g., allergenic and/or carcinogenic properties, both, and the like.

In stage 6 (recommendation assessment), “best practice” templates are matched using case-specific optimized weights against the recommendation. In an embodiment, if matching criteria are not met by this matching, the inputs used to generate the recommendation may be resubmitted for stage 2 processing by a different rules cluster.

FIG. 6 illustrates a flow diagram 600 illustrating a process for extracting semantic concepts and relationships therebetween which may be utilized for creation of a knowledge graph according to another embodiment. The process depicted in FIG. 6 may be utilized as part of a model which takes unstructured text data (e.g., literature, product reviews, etc.) and uses natural language processing and machine learning techniques in order to extract concepts and relationships which may be utilized to inform product recommendations based on user preferences and needs.

In FIG. 6, input data 610 which may include references to attributes and care practices or portions thereof is analyzed in order to identify one or more input data examples 620. As depicted in FIG. 6, the input data may include structured or unstructured data such as, but not limited to, textual or other content from social networks, search engines, scientific journals, textbooks, product reviews, news, survey data, regulatory agency data, product interactions data, medical data, databases, combinations thereof, and the like. The input data examples 620 include portions of the input data such as, but not limited to, text data for particular product reviews.

From the input data examples 620, dense embedding 630 is performed in order to extract features to be input to one or more rules clusters of a hierarchical rule base 640 during a NLP/ML process. The outputs of the hierarchical rule base 640 are utilized in order to generate a set of extracted concepts and relationships 650 between the extracted concepts, which in turn are utilized to create a mapping between such concepts in order to create a knowledge graph as further described above.

It should be noted that various embodiments may be discussed with respect to hair in the context of human hair, but that the disclosed embodiments are not limited as such. Various disclosed embodiments may be equally applicable to fur or other health-related attributes of animals (e.g., fur of pets such as dogs and cats) without departing from the scope of the disclosure. Moreover, various disclosed embodiments are described with respect to analyzing discrete attributes of users, but a person having ordinary skill in the art would readily understand that at least some embodiments can be implemented using attributes of non-user subjects such as other humans or animals without departing from the scope of the disclosure.

It should also be noted that various embodiments are described with respect to mapping hair attributes to ingredients and profiling hair of users, but a person having ordinary skill in the art would readily recognize that the disclosed embodiments may be likewise applicable to identifying connections between particular skin attributes and discrete components of skin care practices or products, or similarly for other discrete health-related attributes and healthcare products or practices, without departing from the scope of the disclosure.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for discretizing connections of semantically defined attributes using multi-stage machine learning, comprising:
identifying a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices;
mapping between semantic concepts of the plurality of semantic concepts, wherein mapping between the semantic concepts further comprises applying a first machine learning model trained to identify correlations between the plurality of semantic concepts with respect to the attributes and care practices identified within the population data;
creating a knowledge graph including a plurality of nodes representing the plurality of semantic concepts and a plurality of edges connecting the plurality of nodes based on the mapping;
applying a second machine learning model to visual content for a user in order to identify a subset of the attribute semantic concepts for the user, wherein the second machine learning model is trained to identify attribute semantic concepts of the plurality of attribute semantic concepts shown in the visual content; and
querying the knowledge graph based on the identified subset of the attribute semantic concepts output for the second user, wherein the knowledge graph returns at least one care practice component semantic concept connected to the queried subset of the attribute semantic concepts.

2. The method of claim 1, further comprising:
generating at least one recommendation based on the at least one discrete component of care practices returned by the knowledge graph.

3. The method of claim 2, wherein generating the at least one recommendation further comprises:
identifying at least one care practice including at least a portion of the care practice components represented by the at least one care practice component semantic concept returned by the knowledge graph, wherein the at least one recommendation is generated based on the identified at least one care practice.

4. The method of claim 3, wherein generating the at least one recommendation further comprises:
generating a first recommendation based on the identified at least one care practice;
logging progress of the user with respect to the at least one care practice as used by the user, wherein the progress is logged using inputs from the user defined with respect to the plurality of semantic concepts; and
generating a second recommendation based on the first recommendation and the logged progress.

5. The method of claim 4, wherein the second recommendation is generated based further on a target for the user, wherein the target is defined with respect to at least one of the plurality of attribute semantic concepts.

6. The method of claim 5, wherein the target is determined by applying at least one interaction rule based on at least one portion of content viewed by the user during an exploration and data indicating user interactions with the at least one portion of content during the exploration.

7. The method of claim 4, further comprising:
updating the knowledge graph based on the logged progress.

8. The method of claim 2, wherein generating the at least one recommendation further comprises:
applying a preference engine to a plurality of potential recommendations, wherein the preference engine is configured to determine whether each of the plurality of potential recommendations is in line with at least one preference of the user; and
selecting the at least one recommendation from among the plurality of potential recommendations based on output of the preference engine.

9. The method of claim 1, further comprising:
generating a profile for the user based on the plurality of attribute semantic concepts for the user and the knowledge graph.

10. The method of claim 1, further comprising:
determining a confidence level for the subset of the attribute semantic concepts for the user;
determining that the confidence level is below a threshold;
requesting at least one additional input from the user, wherein the requested at least one additional input includes at least one of: a confirmation or a rejection of each of the subset of the attribute semantic concepts for the user; and
updating the subset of the attribute semantic concepts for the user, wherein the knowledge graph is queried using the updated subset.

11. The method of claim 1, further comprising:
defining the plurality of semantic concepts such that each of the attribute semantic concepts is a data object including at least one first term collectively representing a discrete attribute of a respective condition and each of the care practice component semantic concepts is a data object including at least one second term collectively representing a discrete component of a care practice.

12. The method of claim 1, wherein the plurality of attribute semantic concepts includes semantic concepts representing hair attributes, wherein the plurality of care practice component semantic concepts includes semantic concepts representing discrete ingredients of hair care products used for treating hair.

13. A non-transitory computer readable medium having stored thereon instructions for causing a processing circuitry to execute a process, the process comprising:
identifying a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices;
mapping between semantic concepts of the plurality of semantic concepts, wherein mapping between the semantic concepts further comprises applying a first machine learning model trained to identify correlations between the plurality of semantic concepts with respect to the attributes and care practices identified within the population data;
creating a knowledge graph including a plurality of nodes representing the plurality of semantic concepts and a plurality of edges connecting the plurality of nodes based on the mapping;
applying a second machine learning model to visual content for a user in order to identify a subset of the attribute semantic concepts for the user, wherein the second machine learning model is trained to identify attribute semantic concepts of the plurality of attribute semantic concepts shown in the visual content; and
querying the knowledge graph based on the identified subset of the attribute semantic concepts output for the second user, wherein the knowledge graph returns at least one care practice component semantic concept connected to the queried subset of the attribute semantic concepts.

14. A system for discretizing connections of semantically defined attributes using multi-stage machine learning, comprising:
a processing circuitry; and
a memory, the memory containing instructions that, when executed by the processing circuitry, configure the system to:
identify a plurality of attributes and a plurality of care practices indicated within population data, wherein the attributes and the care practices are defined via a plurality of semantic concepts including a plurality of attribute semantic concepts representing known discrete attributes of conditions and a plurality of care practice component semantic concepts representing known discrete components of care practices;
map between semantic concepts of the plurality of semantic concepts, wherein the system is further configured to apply a first machine learning model trained to identify correlations between the plurality of semantic concepts with respect to the attributes and care practices identified within the population data;
create a knowledge graph including a plurality of nodes representing the plurality of semantic concepts and a plurality of edges connecting the plurality of nodes based on the mapping;
apply a second machine learning model to visual content for a user in order to identify a subset of the attribute semantic concepts for the user, wherein the second machine learning model is trained to identify attribute semantic concepts of the plurality of attribute semantic concepts shown in the visual content; and
query the knowledge graph based on the identified subset of the attribute semantic concepts output for the second user, wherein the knowledge graph returns at least one care practice component semantic concept connected to the queried subset of the attribute semantic concepts.

15. The system of claim 14, wherein the system is further configured to:
generate at least one recommendation based on the at least one discrete component of care practices returned by the knowledge graph.

16. The system of claim 15, wherein the system is further configured to:
identify at least one care practice including at least a portion of the care practice components represented by the at least one care practice component semantic concept returned by the knowledge graph, wherein the at least one recommendation is generated based on the identified at least one care practice.

17. The system of claim 16, wherein the system is further configured to:
generate a first recommendation based on the identified at least one care practice;
log progress of the user with respect to the at least one care practice as used by the user, wherein the progress is logged using inputs from the user defined with respect to the plurality of semantic concepts; and
generate a second recommendation based on the first recommendation and the logged progress.

18. The system of claim 17, wherein the second recommendation is generated based further on a target for the user, wherein the target is defined with respect to at least one of the plurality of attribute semantic concepts.

19. The system of claim 18, wherein the target is determined by applying at least one interaction rule based on at least one portion of content viewed by the user during an exploration and data indicating user interactions with the at least one portion of content during the exploration.

20. The system of claim 17, wherein the system is further configured to:
update the knowledge graph based on the logged progress.

21. The system of claim 15, wherein the system is further configured to:
apply a preference engine to a plurality of potential recommendations, wherein the preference engine is configured to determine whether each of the plurality of potential recommendations is in line with at least one preference of the user; and
select the at least one recommendation from among the plurality of potential recommendations based on output of the preference engine.

22. The system of claim 14, wherein the system is further configured to:
generate a profile for the user based on the plurality of attribute semantic concepts for the user and the knowledge graph.

23. The system of claim 14, wherein the system is further configured to:
determine a confidence level for the subset of the attribute semantic concepts for the user;
determine that the confidence level is below a threshold;
request at least one additional input from the user, wherein the requested at least one additional input includes at least one of: a confirmation or a rejection of each of the subset of the attribute semantic concepts for the user; and
update the subset of the attribute semantic concepts for the user, wherein the knowledge graph is queried using the updated subset.

24. The system of claim 14, wherein the system is further configured to:
define the plurality of semantic concepts such that each of the attribute semantic concepts is a data object including at least one first term collectively representing a discrete attribute of a respective condition and each of the care practice component semantic concepts is a data object including at least one second term collectively representing a discrete component of a care practice.

25. The system of claim 14, wherein the plurality of attribute semantic concepts includes semantic concepts representing hair attributes, wherein the plurality of care practice component semantic concepts includes semantic concepts representing discrete ingredients of hair care products used for treating hair.
